(19) United States
(12) Patent Application Publication
(10) Pub. No.: US 2018/0110485 A1
(43) Pub. Date: Apr. 26, 2018

(54) SUBJECT FIXING DEVICE AND MEDICAL IMAGE DIAGNOSTIC APPARATUS

(71) Applicant: Hitachi, Ltd., Tokyo (JP)

(72) Inventors: Masaya TAMAOKI, Tokyo (JP); Kenya SAKANAKA, Tokyo (JP); Yuta INOUE, Tokyo (JP)

(73) Assignee: Hitachi, Ltd., Tokyo (JP)

(21) Appl. No.: 15/562,876

(22) PCT Filed: Feb. 18, 2016
(86) PCT No.: PCT/JP2016/054651

§ 371 Date: Sep. 28, 2017

(30) Foreign Application Priority Data
Mar. 30, 2015 (JP) 2015-068685

(51) Int. Cl. A61B 6/04; A61B 5/055

(52) U.S. Cl. CPC A61B 6/0421; A61B 5/0555

(57) ABSTRACT

In order to more reliably prevent a runner from being disengaged from a rail during using of a fixation band, a subject fixing device of the present invention includes a runner includes a runner main body that is accommodated inside a rail, and a band connection portion that protrudes out of the rail and is connected to the fixation band, and a rail includes a rail main body that slidably holds the runner main body, and first and second attachment portions that protrude outwards from the rail main body, and are disposed with an end of the fixation base interposed therebetween so as to be attached to the fixation base, and in which the rail main body is provided with a slit which allows the band connection portion to pass therethrough, and prevents the runner main body from passing therethrough, along a longitudinal direction of the rail main body.

### TECHNICAL FIELD

The present invention relates to a subject fixing device which fixes a subject to a fixation base provided in, for example, an X-ray CT apparatus or an MRI apparatus, and a medical image diagnostic apparatus using the same.

### BACKGROUND ART

In a subject fixing device of the related art, a fixation belt is attached to a fixation base to which a subject is fixed, via an attachment member. The attachment member is attached to the fixation base via a surface fastener. A hook-shaped fitting portion is provided at an end of the fixation belt on the attachment member side. The attachment member is provided with a receiving portion to which the fitting portion is fitted. The fitting portion can be slid in a longitudinal direction of the fixation base along the receiving portion in a state of being fitted to the receiving portion (refer to PTL 1).

### CITATION LIST

### Patent Literature

PTL 1: Japanese Patent No. 4521072

### SUMMARY OF INVENTION

### Technical Problem

In the subject fixing device of the related art as described above, since the fitting portion is only fitted to the receiving portion, the fitting portion may be disengaged from the receiving portion in a case where the fixation belt is pulled with strong force. In a case where the receiving portion is located at the end of the attachment member in the longitudinal direction, if the fixation belt is pulled obliquely with respect to the attachment member, the receiving portion may be detached from the end of the fitting portion.

The present invention has been made in order to solve the problem, and an object thereof is to provide a subject fixing device which can more reliably prevent a runner from being disengaged from a rail during using of a fixation band, and a medical image diagnostic apparatus using the subject fixing device.

### Solution to Problem

According to the present invention, there is provided a subject fixing device including a rail that is attached to a fixation base; and a runner that is combined with the rail slidably along a longitudinal direction of the rail, and is connected to a fixation band for fixing a subject to the fixation base, in which the runner includes a runner main body that is accommodated inside the rail, and a band connection portion that protrudes out of the rail and is connected to the fixation band, in which the rail includes a rail main body that slidably holds the runner main body, and first and second attachment portions that protrude from the rail main body, and are disposed with an end of the fixation base interposed therebetween so as to be disposed to the fixation base, in which the rail main body is provided with a slit which allows the band connection portion to pass therethrough, and prevents the runner main body from passing therethrough, along a longitudinal direction of the rail main body, in which both ends of the slit in the longitudinal direction of the rail main body are closed, and in which an opening through which the runner passes is provided at a portion of the rail main body facing the slit between the first attachment portion and the second attachment portion.

### Advantageous Effects of Invention

The subject fixing device of the present invention can more reliably prevent a runner from being disengaged from a rail during using of a fixation band.

### BRIEF DESCRIPTION OF DRAWINGS

FIG. 1 is a block configuration diagram illustrating a part of an X-ray CT apparatus according to Embodiment 1 of the present invention.

FIG. 2 is a perspective view illustrating principal portions of a bed illustrated in FIG. 1.

FIG. 3 is a sectional view illustrating a state in the middle of a subject fixing device illustrated in FIG. 2 being attached to a top plate.

FIG. 4 is a perspective view illustrating the subject fixing device illustrated in FIG. 3.

FIG. 5 is a perspective view illustrating a state in which a runner in FIG. 4 is detached from a rail.

FIG. 6 is a diagram illustrating a subject fixing device according to Embodiment 2 of the present invention.

FIG. 7 is a diagram illustrating a state in which a runner in FIG. 6 is detached from a rail.

FIG. 8 is a diagram illustrating a subject fixing device according to Embodiment 3 of the present invention.

FIG. 9 is a diagram illustrating a state in which a runner in FIG. 8 is detached from a rail.

### DESCRIPTION OF EMBODIMENTS

A subject fixing device of the present invention includes a rail that is attached to a fixation base; and a runner that is combined with the rail slidably along a longitudinal direction of the rail, and is connected to a fixation band for fixing a subject to the fixation base, in which the runner includes a runner main body that is accommodated inside the rail, and a band connection portion that protrudes out of the rail and is connected to the fixation band, in which the rail includes a rail main body that slidably holds the runner main body, and first and second attachment portions that protrude from the rail main body, and are disposed with an end of the fixation base interposed therebetween so as to be disposed to the fixation base, in which the rail main body is provided with a slit which allows the band connection portion to pass therethrough, and prevents the runner main body from passing therethrough, along a longitudinal direction of the rail main body, in which both ends of the slit in the longitudinal direction of the rail main body are closed, and in which an opening through which the runner passes is provided at a portion of the rail main body facing the slit between the first attachment portion and the second attachment portion.

A shape of a section of the rail main body which is perpendicular to the longitudinal direction is a C shape, and the opening is provided over the entire longitudinal direction of the rail main body.

The subject fixing device further includes a locking pin, the rail main body is provided with a plurality of rail pin holes through which the locking pin penetrates, with gaps therebetween in the longitudinal direction of the rail, and the runner main body is provided with a runner pin hole into which the locking pin is inserted.

The subject fixing device further includes a holder that is attached to the rail by embracing the rail main body across the slit, and prevents a width of the slit from increasing.

According to the present invention, there is provided a medical image diagnostic apparatus including the subject fixing device.

Hereinafter, embodiments of the present invention will be described with reference to the drawings.

### Embodiment 1

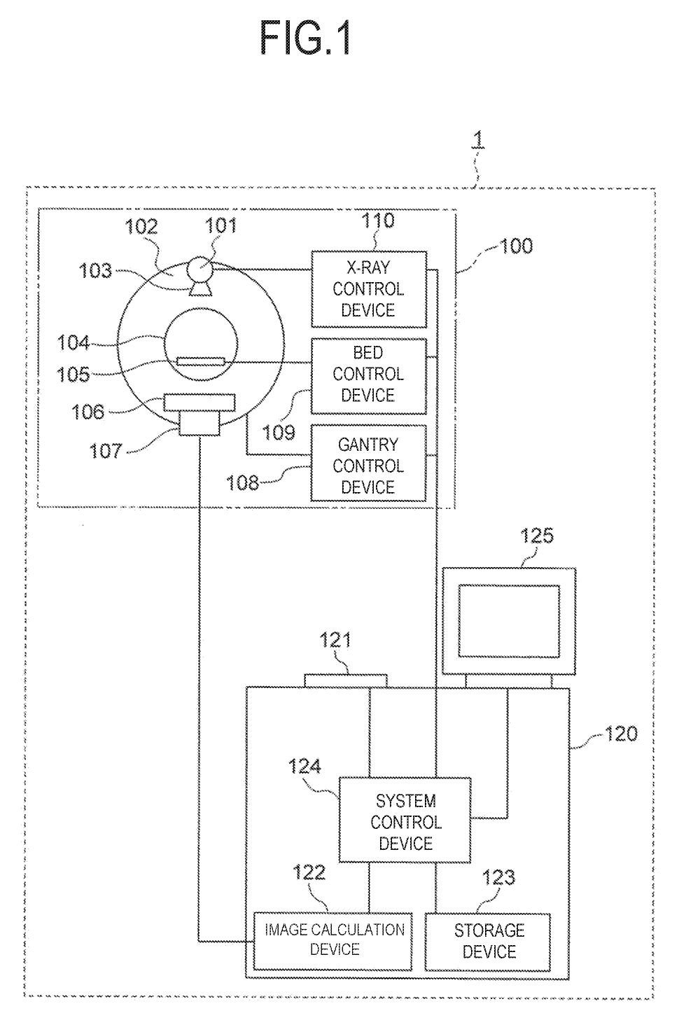

FIG. 1 is a block configuration diagram illustrating a part of an X-ray CT apparatus 1 according to Embodiment 1 of the present invention. The X-ray CT apparatus 1 includes a scan gantry portion 100 and an operation console 120.

The scan gantry portion 100 includes an X-ray tube 101, a rotation disk 102, a collimator 103, a bed 105, an X-ray detector 106, a data collecting device 107, a gantry control device 108, a bed control device 109, and an X-ray control device 110.

The X-ray tube 101 is a device which irradiates a subject placed on the bed 105 with X-rays. The collimator 103 is a device which restricts an irradiation range of X-rays applied from the X-ray tube 101. The X-ray tube 101 and the X-ray detector 106 are mounted on the rotation disk 102. The rotation disk 102 is provided with an opening 104 into which the subject placed on the bed 105 is inserted. The rotation disk 102 is rotated about the subject.

The X-ray detector 106 is disposed to oppose the X-ray tube 101. The X-ray detector 106 is a device which detects X-rays having been transmitted through the subject, and measures a spatial distribution of the transmitted X-rays. The X-ray detector 106 is a device in which a plurality of X-ray detection elements are arranged along a rotation direction of the rotation disk 102, or are two-dimensionally arranged in the rotation direction and a rotation axis direction of the rotation disk 102.

The data collecting device 107 is a device which collects an X-ray dose detected by the X-ray detector 106 as digital data. The gantry control device 108 is a device which controls rotation of the rotation disk 102. The bed control device 109 is a device which controls movement in an upward-and-downward direction and a front-and-rear direction of the bed 105. The X-ray control device 110 is a device which controls power which is input to the X-ray tube 101.

The operation console 120 includes an input device 121, an image calculation device 122, a storage device 123, a system control device 124, and a display device 125. The input device 121 is a device for inputting the name of a subject, the examination date and time, scanning conditions, and the like. The input device 121 is provided with, for example, a keyboard and a pointing device.

The image calculation device 122 is a device which performs a calculation process on measured data transmitted from the data collecting device 107, and reconstructs a CT image. The display device 125 is a device which displays a CT image created by the image calculation device 122. As the display device 125, for example, a cathode-ray tube (CRT) or a liquid crystal display is used.

The storage device 123 is a device which stores the data collected by the data collecting device 107 and image data of the CT image created by the image calculation device 122. As the storage device 123, a hard disk drive (HDD) or the like is used. The system control device 124 is a device which controls the devices 121, 122, 123 and 125, the gantry control device 108, the bed control device 109, and the X-ray control device 110.

Functions of the control device, the calculation device, and the storage unit may be realized by using, for example, a plurality of computers.

The X-ray control device 110 controls power which is input to the X-ray tube 101 on the basis of scanning conditions which are input from the input device 121, especially, an X-ray tube voltage or an X-ray tube current. Consequently, the X-ray tube 101 irradiates the subject with X-rays corresponding to the scanning conditions. The X-ray detector 106 detects X-rays which are applied from the X-ray tube 101 and are transmitted through the subject with a plurality of X-ray detection elements, and measures a distribution of the transmitted X-rays.

The gantry control device 108 rotates the rotation disk 102 on the basis of scanning conditions which are input from the input device 121, especially, a rotation speed and the like. The bed control device 109 operates the bed 105 on the basis of scanning conditions which are input from the input device 121, especially, a spiral pitch and the like.

X-ray irradiation from the X-ray tube 101 and measurement of a transmitted X-ray distribution performed by the X-ray detector 106 are repeatedly performed along with rotation of the rotation disk 102, and thus projection data from various angles is acquired. The acquired projection data is transmitted to the image calculation device 122. The image calculation device 122 performs an inverse projection process on the transmitted projection data so as to reconstruct a CT image. The reconstructed CT image is displayed on the display device 125.

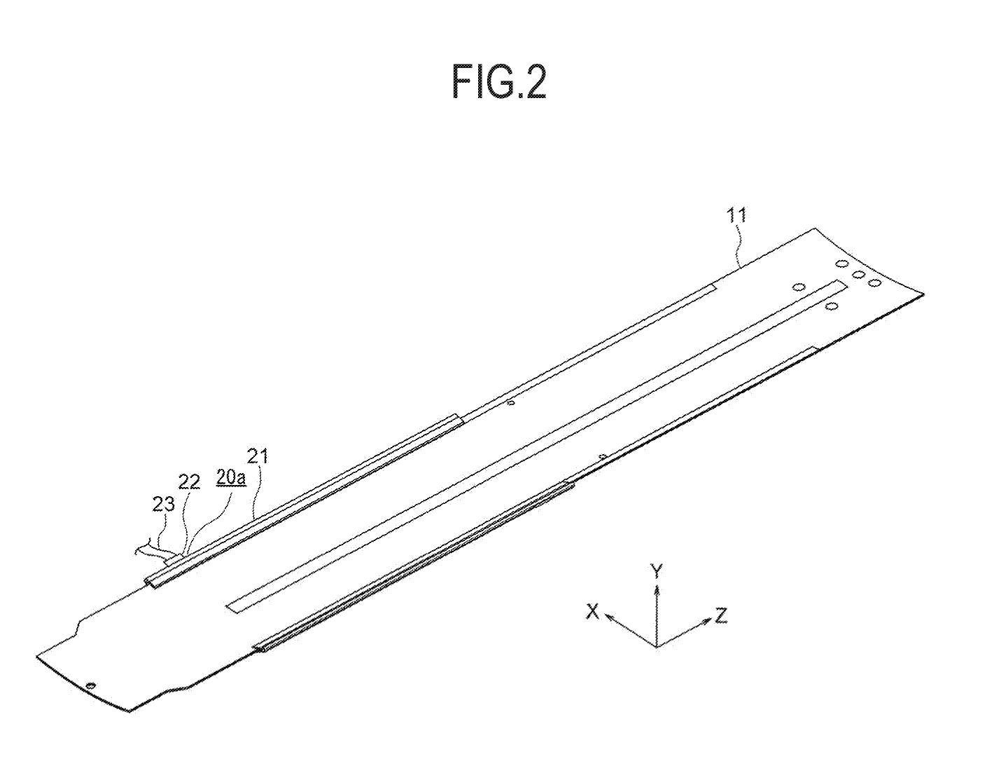

FIG. 2 is a perspective view illustrating principal portions of the bed 105 illustrated in FIG. 1. The bed 105 has a top plate 11 as a fixation base to which a subject is fixed. A planar shape of the top plate 11 is a rectangular shape in which a dimension in a front-and-rear direction (Z axis direction in the figure) is larger than a dimension in a width direction (X axis direction in the figure). In other words, a longitudinal direction of the top plate 11 is the Z axis direction in the figure, and is a body axis direction of a subject on the top plate 11.

A pair of subject fixing devices 20a for fixing a subject to the top plate 11 are provided at both ends of the top plate 11 in the width direction. Each of the pair of subject fixing devices 20a includes a rail 21 attached to the top plate 11, a runner 22 which is slidably combined with the rail 21 along a longitudinal direction of the rail 21, and a flexible fixation band 23 connected to the runner 22.

The rail 21 is continuously disposed along the longitudinal direction of the top plate 11. In other words, the longitudinal direction of the rail 21 is the same as the longitudinal direction of the top plate 11. A dimension of the runner 22 in the longitudinal direction of the rail 21 is smaller than a dimension of the rail 21 in the longitudinal direction. The fixation bands 23 on both sides of the top plate 11 in the width direction are coupled to each other, and thus a subject is fixed to the top plate 11.

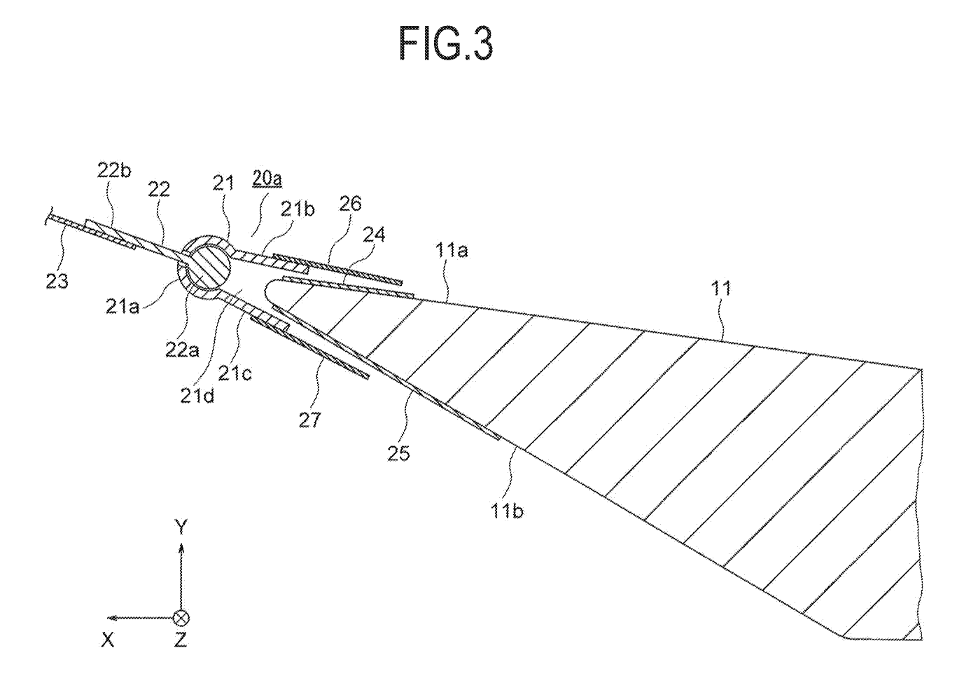

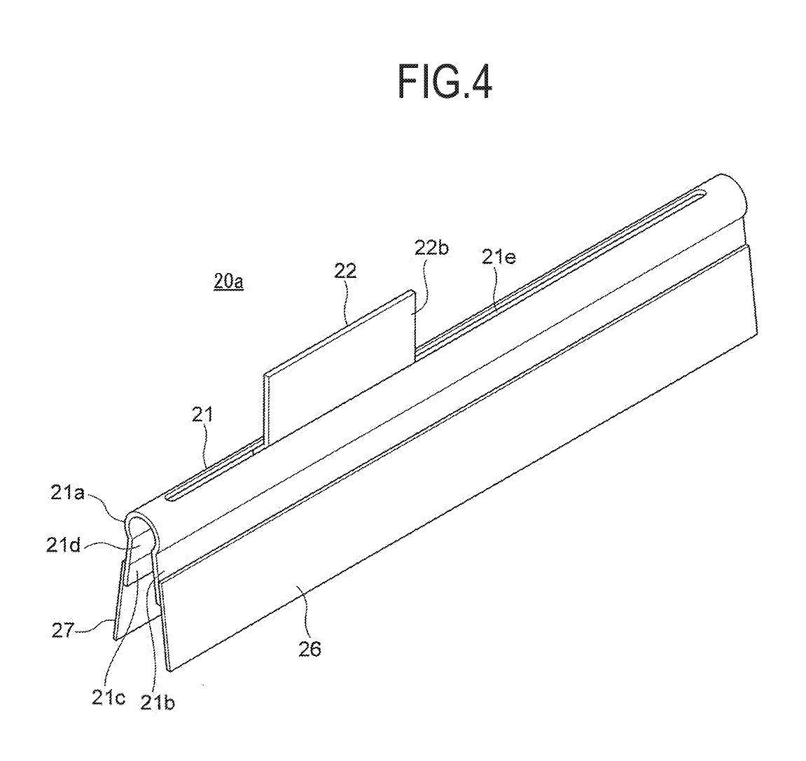

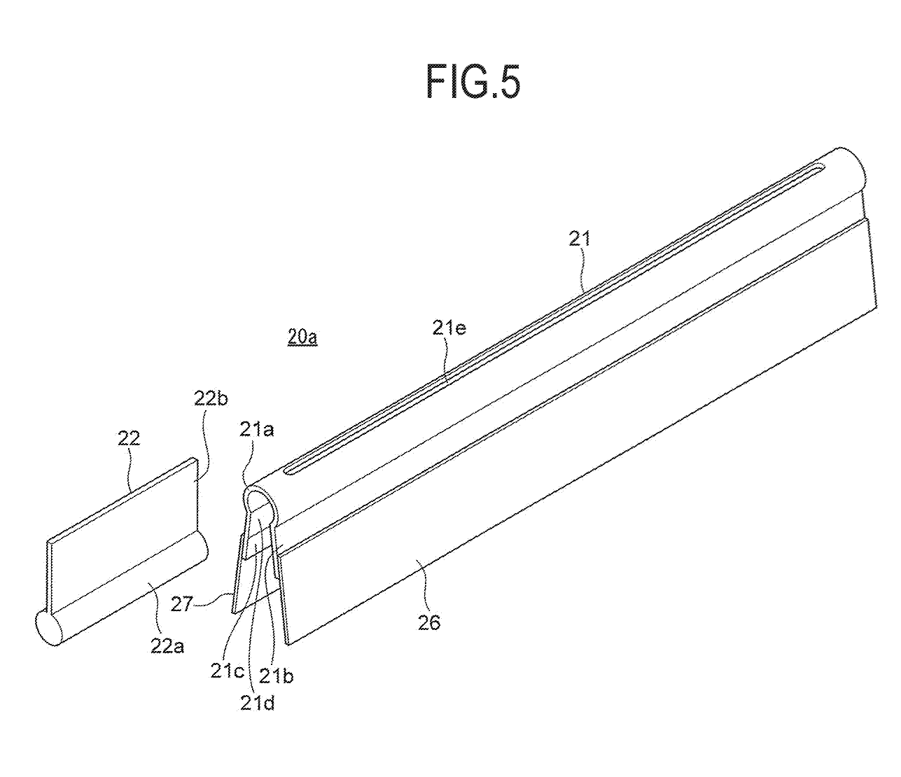

FIG. 3 is a sectional view illustrating a state in the middle of the subject fixing device 20a in FIG. 2 being attached to the top plate 11, and illustrates a section of the top plate 11 which is perpendicular to the longitudinal direction. FIG. 4 is a perspective view illustrating the subject fixing device 20a in FIG. 3, and FIG. 5 is a perspective view illustrating a state in which the runner 22 in FIG. 4 is detached from the rail 21.

The rail 21 and the runner 22 are made of, for example, a resin such as polypropylene.

The runner 22 includes a cylindrical runner main body 22a which is accommodated inside the rail 21, and a tabular band connection portion 22b which protrudes out of the rail 21. The fixation band 23 is connected to the band connection portion 22b, for example, through sewing.

The rail 21 includes a rail main body 21a having a C-shaped section which slidably holds the runner main body 22a, a tabular first attachment portion 21b which protrudes from the rail main body 21a, and a tabular second attachment portion 21c which protrudes from the rail main body 21a with a gap in a circumferential direction of the rail main body 21a with respect to the first attachment portion 21b.

In a section of the rail 21 which is perpendicular to the longitudinal direction, the first attachment portion 21b protrudes outward in a diameter direction from a first end of the rail main body 21a, and the second attachment portion 21c protrudes outward in the diameter direction from a second end of the rail main body 21a. The first and second attachment portions 21b and 21c are continuously provided in the longitudinal direction of the rail main body 21a, that is, over the entire longitudinal direction of the rail 21.

An opening 21d through which the entire runner 22 passes is provided at a portion of the rail main body 21a between the first attachment portion 21b and the second attachment portion 21c. The opening 21d is provided over the entire longitudinal direction of the rail main body 21a. A width dimension of the opening 21d, that is, a distance between the ends of the first and second attachment portions 21b and 21c on the rail main body 21a side is larger than a diameter of the runner main body 22a, or becomes larger than the diameter of the runner main body 22a by elastically deforming the rail main body 21a.

The rail main body 21a is provided with a slit 21e. The slit 21e is provided along the longitudinal direction of the rail 21. The opening 21d faces the slit 21e. A width dimension of the slit 21e is larger than a thickness dimension of the band connection portion 22b, and is smaller than a diameter of the runner main body 22a. Consequently, the slit 21e allows the band connection portion 22b to pass therethrough, and prevents the runner main body 22a from passing therethrough.

The slit 21e is provided at only the intermediate portion except for both ends of the rail main body 21a in the longitudinal direction. In other words, both ends of the slit 21e in the longitudinal direction of the rail main body 21a are closed. Thus, the runner 22 cannot be inserted into the rail 21 from the end of the rail 21 in the longitudinal direction, and can be inserted from only the opening 21d.

A first top plate surface fastener 24 is fixed to an upper surface 11a at the end of the top plate 11 in the width direction. A second top plate surface fastener 25 is fixed to a lower surface 11b at the end of the top plate 11 in the width direction. The first and second top plate surface fasteners 24 and 25 are fixed to the top plate 11, for example, through adhesion.

The first attachment portion 21b is connected to a first rail surface fastener 26 coupled to the first top plate surface fastener 24. The second attachment portion 21c is connected to a second rail surface fastener 27 coupled to the second top plate surface fastener 25. The first and second rail surface fasteners 26 and 27 are respectively connected to the first and second attachment portions 21b and 21c, for example, through sewing.

The rail 21 is disposed so that the end of the top plate 11 in the width direction is interposed between the first and second attachment portions 21b and 21c. The first attachment portion 21b is attachably and detachably attached to the top plate 11 via the first top plate surface fastener 24 and the first rail surface fastener 26. The second attachment portion 21c is attachably and detachably attached to the top plate 11 via the second top plate surface fastener 25 and the second rail surface fastener 27.

In this subject fixing device 20a, the slit 21e whose both ends are closed is provided in the rail main body 21a, and the opening 21d is provided at the portion of the rail main body 21a facing the slit 21e between the first attachment portion 21b and the second attachment portion 21c. Thus, movement of the runner 22 in the longitudinal direction of the rail 21 is restricted at both ends of the slit 21e by the rail main body 21a.

In a state in which the rail 21 is attached to the top plate 11, movement of the runner 22 in a direction of coming off from the rail 21 is also restricted by the first and second attachment portions 21b and 21c and the width direction end of the top plate 11.

Since both ends of the slit 21e are closed, and the portions of the rail main body 21a adjacent to both ends of the slit 21e are connected to each other, such deformation of the rail main body 21a that the width of the slit 21e increases is prevented.

With the above-described configuration, the runner 22 can be more reliably prevented from being disengaged from the rail 21 during using of the fixation band 23.

If the rail 21 is detached from the top plate 11, the runner 22 can be easily extracted from the rail 21, and thus the fixation band 23 can also be easily replaced.

Since the rail 21 is attached to the top plate 11 via the first top plate surface fastener 24, the second top plate surface fastener 25, the first rail surface fastener 26, and the second rail surface fastener 27, the rail 21 is hardly disengaged from the top plate 11 even if the fixation band 23 is pulled in a direction in which shearing force acts on between the first top plate surface fastener 24 and the first rail surface fastener 26 or between the second top plate surface fastener 25 and the second rail surface fastener 27, during using of the fixation band 23.

Force causing the width of the slit 21e to be increased can be prevented from being applied to the rail main body 21a by the flexible movement of the first rail surface fastener 26 and the second rail surface fastener 27.

The opening 21d is provided over the entire longitudinal direction of the rail main body 21a, and thus a configuration is simple, and manufacturing is easy.

Since a section of the rail main body 21a has a C shape, and an outer shape of a section of the runner main body 22a is a circular shape, the runner 22 can be smoothly slid with respect to the rail 21.

### Embodiment 2

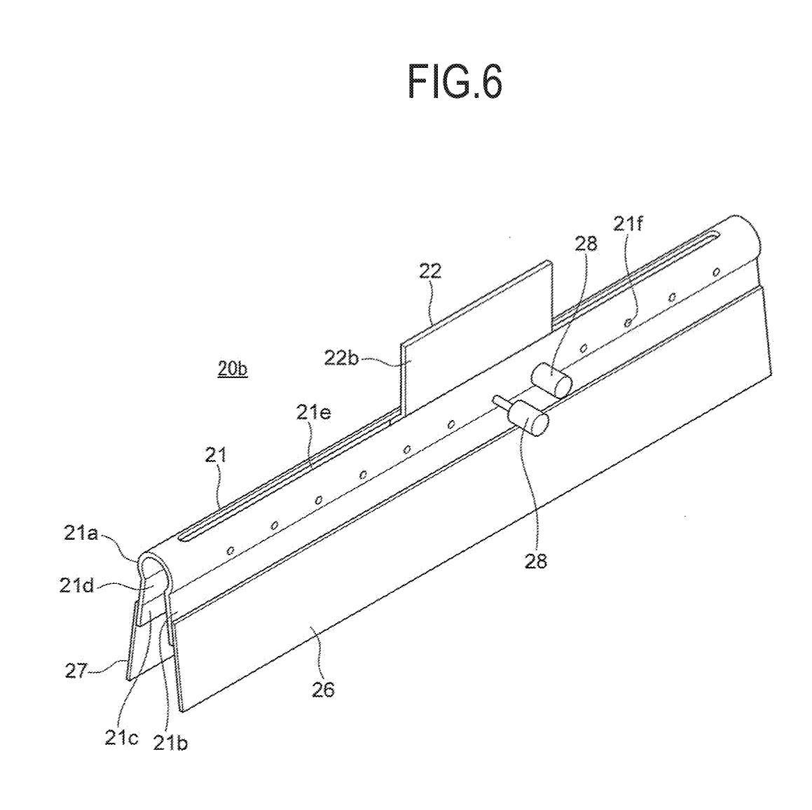

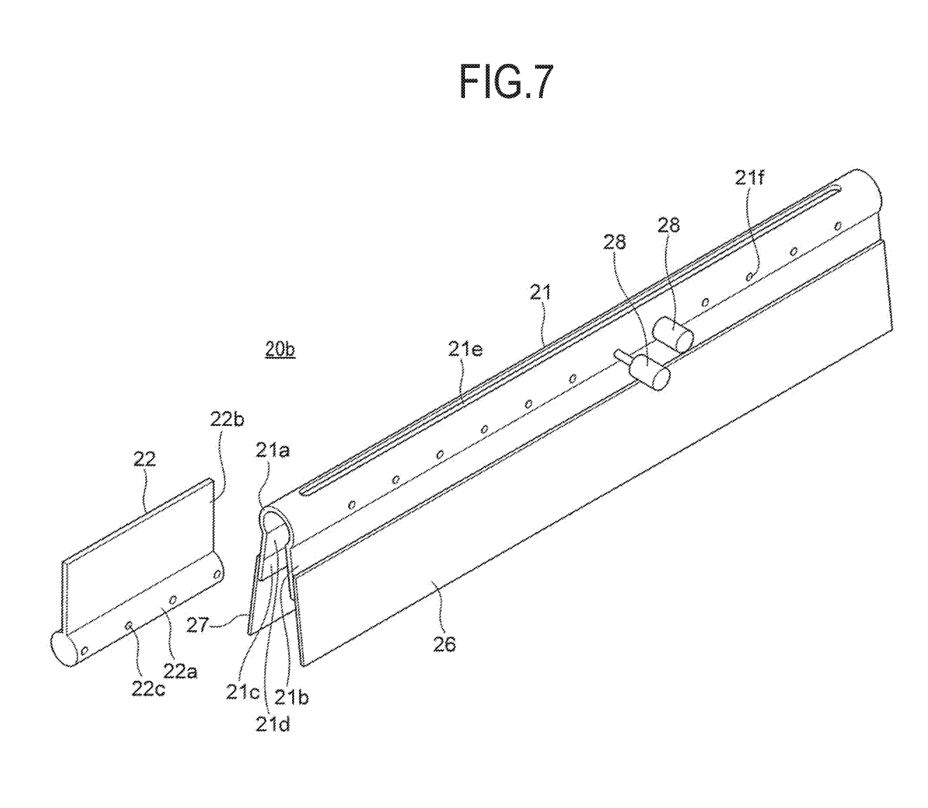

Next, FIG. 6 is a diagram illustrating a pair of subject fixing devices 20b for fixing a subject to the top plate 11 according to Embodiment 2 of the present invention, and FIG. 7 is a state in which the runner 22 in FIG. 6 is detached from the rail 21. The subject fixing device 20b of Embodiment 2 further includes a plurality of locking pins 28 in addition to the configuration of Embodiment 1.

A plurality of rail pin holes 21f through which the locking pins 28 penetrate are provided on the side surface of the rail main body 21a with gaps between each other in the longitudinal direction of the rail 21. The rail pin holes 21f are disposed at the same interval linearly in parallel to the longitudinal direction of the rail 21.

A plurality of runner pin holes 22c into which the locking pins 28 are inserted are provided on the side surface of the runner main body 22a. The runner pin holes 22c are disposed at the same interval as the interval of the rail pin holes 21f linearly in parallel to the longitudinal direction of the rail 21 when the runner 22 is combined with the rail 21. Other constituent elements of the subject fixing device 20b and the entire configuration of the X-ray CT apparatus are the same as those in Embodiment 1.

The same effect as in Embodiment 1 can also be achieved by using the subject fixing device 20b. Since a position of the runner 22 is adjusted, and then the locking pins 28 are inserted into the rail pin holes 21f and the runner pin holes 22c, the runner 22 can be more reliably prevented from being disengaged from the rail 21 during using of the fixation band 23. The runner 22 can be prevented from being moved in the longitudinal direction of the rail 21, and thus workability can be improved.

The intervals and the number of the pin holes 21f and 22c are not particularly limited.

The pin holes 21f and 22c are not necessarily required to be disposed at the same interval.

### Embodiment 3

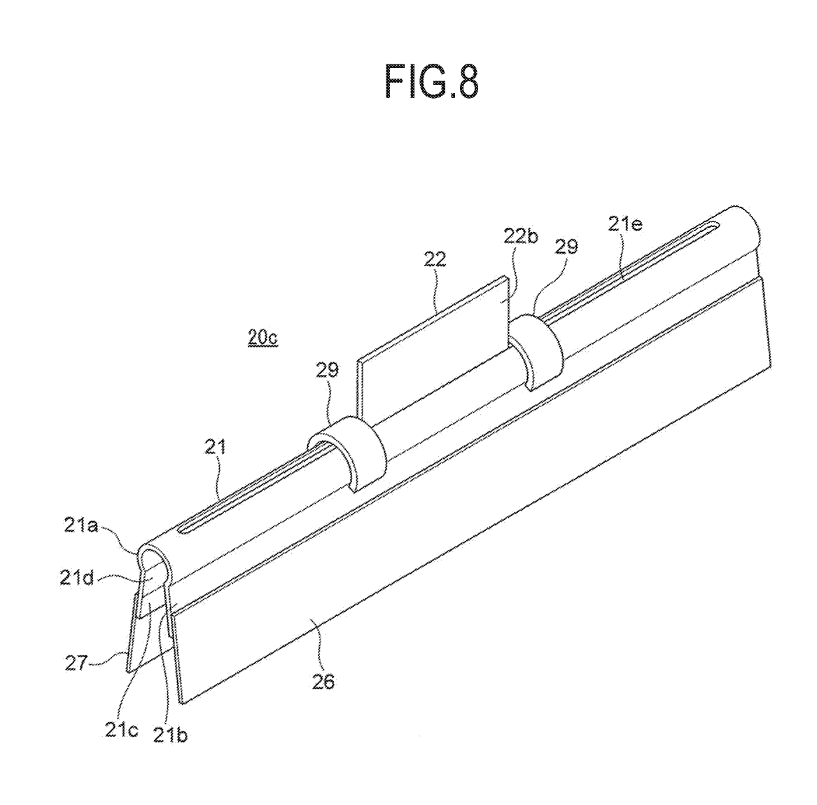

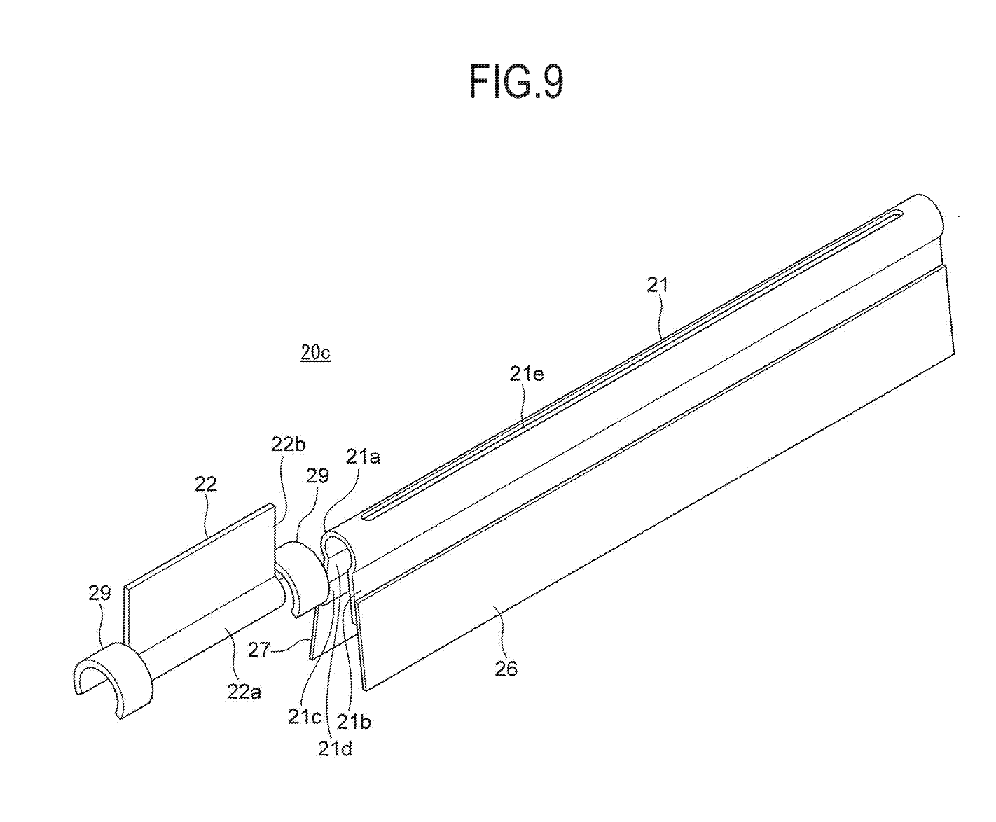

Next, FIG. 8 is a diagram illustrating a pair of subject fixing devices 20c for fixing a subject to the top plate 11 according to Embodiment 3 of the present invention, and FIG. 9 is a state in which the runner 22 in FIG. 8 is detached from the rail 21. The subject fixing device 20c of Embodiment 3 further includes a pair of holders 29 having a C-shaped section in addition to the configuration of Embodiment 1.

Each of the holders 29 is attached to the rail 21 by embracing the rail main body 21a across the slit 21e. Consequently, the holders 29 prevent the width of the slit 21e from increasing. The holders 29 are in contact with the band connection portion 22b at both ends of the band connection portion 22b in the longitudinal direction of the rail 21. Other constituent elements of the subject fixing device 20c and the entire configuration of the X-ray CT apparatus are the same as those in Embodiment 1.

The same effect as in Embodiment 1 can also be achieved by using the subject fixing device 20c. Since the holders 29 are attached to the rail 21, the width of the slit 21e can be prevented from increasing, and thus the runner 22 can be more reliably prevented from being disengaged from the rail 21 via the slit 21e.

Since the holders 29 tighten the rail main body 21a with a certain degree of force in a state in which the holders 29 are attached to the rail 21, the runner 22 can be prevented from being moved in the longitudinal direction of the rail 21, and thus workability can be improved.

The number of holders 29 may be one or three or more.

The holders 29 may be disposed with respect to the band connection portion 22b with a gap therebetween in the longitudinal direction of the rail 21.

The holders 29 of Embodiment 3 may be attached to the rail 21 of the Embodiment 2.

In the above-described examples, the opening 21d is provided over the entire longitudinal direction of the rail main body 21a, but an opening may be provided in only a part of the rail in the longitudinal direction, and the remaining part of the rail may be closed, as long as the runner can pass through the opening. Consequently, it is possible to increase the strength of the rail.

Two or more runners may be provided in a single rail. In this case, a separate slit may be provided for each runner, and two or more runner may pass through a common slit.

A rail disposed on one side of a fixation base may be divided into two or more portions in a longitudinal direction thereof. In this case, rails adjacent to each other may be continuously disposed, and may be disposed with a gap therebetween.

The first and second attachment portions may be provided intermittently in the longitudinal direction of the rail main body.

A subject fixing device may be provided on only one side of a fixation base in a width direction, and only a component for holding a fixation band is provided on the opposite side.

In the above-described examples, an X-ray CT apparatus has been described as an example of a medical image diagnostic apparatus, but the medical image diagnostic apparatus is not limited thereto, and may be, for example, an MRI apparatus.

### REFERENCE SIGNS LIST

1 X-ray CT apparatus (medical image diagnostic apparatus), 11 top plate (fixation base), 20a, 20b, and 20c subject fixing device, 21 rail, 21a rail main body, 21b first attachment portion, 21c second attachment portion, 21d opening, 21e slit, 21f rail pin hole, 22 runner, 22a runner main body, 22b band connection portion, 22c runner pin hole, fixation band, 28 locking pin, 29 holder

1. A subject fixing device comprising:
a rail that is attached to a fixation base; and
a runner that is combined with the rail slidably along a longitudinal direction of the rail, and is connected to a fixation band for fixing a subject to the fixation base,
wherein the runner includes a runner main body that is accommodated inside the rail, and a band connection portion that protrudes out of the rail and is connected to the fixation band,
wherein the rail includes a rail main body that slidably holds the runner main body, and first and second attachment portions that protrude outwards from the rail main body, and are disposed with an end of the fixation base interposed therebetween so as to be attached to the fixation base,
wherein the rail main body is provided with a slit which allows the band connection portion to pass therethrough, and prevents the runner main body from passing therethrough, along a longitudinal direction of the rail main body,
wherein both ends of the slit in the longitudinal direction of the rail main body are closed, and
wherein an opening through which the runner passes is provided at a portion of the rail main body facing the slit between the first attachment portion and the second attachment portion.

2. The subject fixing device according to claim 1,
wherein a shape of a section of the rail main body which is perpendicular to the longitudinal direction is a C shape, and
wherein the opening is provided over the entire longitudinal direction of the rail main body.

3. The subject fixing device according to claim 1, further comprising:
a locking pin,
wherein the rail main body is provided with a plurality of rail pin holes through which the locking pin penetrates, with gaps therebetween in the longitudinal direction of the rail, and
wherein the runner main body is provided with a runner pin hole into which the locking pin is inserted.

4. The subject fixing device according to claim 1, further comprising:
a holder that is attached to the rail by embracing the rail main body across the slit, and prevents a width of the slit from increasing.

5. A medical image diagnostic apparatus comprising the subject fixing device according to claim 1.
